(19) United States
(12) Patent Application Publication
(10) Pub. No.: US 2020/0013515 A1
(43) Pub. Date: Jan. 9, 2020

(54) DRUG INTERACTION CHECKING TOOL

(71) Applicant: Talis Clinical LLC, Streetsboro, OH (US)

(72) Inventors: Gary Colister, Hudson, OH (US); Bishoy Magdalla, Hudson, OH (US); Giuseppe Saracino, Gainsville, FL (US); William Murphy, Aurora, OH (US); Harish Lecamwasam, Scottsdale, AZ (US); Andrew Bardwell, Brunswick, OH (US)

(21) Appl. No.: 16/503,074

(22) Filed: Jul. 3, 2019

Related U.S. Application Data
(60) Provisional application No. 62/693,764, filed on Jul. 3, 2018.

(51) Int. Cl. G16H 70/40; G16H 10/60

(52) U.S. Cl. CPC G16H 70/40; G16H 20/10; G16H 10/60

(57) ABSTRACT

The present invention is directed to a drug interaction checking tool for use without medication orders or CPOE which includes an interface sub-system to receive messages from healthcare information technology systems with information about the patient such as patient allergies, medications the patient is currently taking, patient factors and co-morbidities, where the interface sub-system also receives messages from a decentralized pharmacy system re withdrawn medications, the clinician, and the patient; a data repository sub-system to store the received data such as patient allergies, medications prescriber, administered or currently in use, patient factors and co-morbidities, medications withdrawn, and the identity of the clinician; access to a medication interactions database; a guidance engine sub-system which performs the medication interaction checks for the patient and generates alerts if a risk of medication errors or adverse reaction is identified; and a notification sub-system which disseminates the alerts to one or more devices and/or systems.

### BACKGROUND OF THE INVENTION

The National Coordinating Council for Medication Error Reporting and Prevention defines a Medication Error (ME) as “any preventable event that may cause or lead to inappropriate medication use or patient harm while the medication is in the control of a healthcare provider, patient or consumer”. As such, a ME can occur due to mistakes at any stage in the medication handling process. An Adverse Drug Event (ADE) is defined as “an injury resulting from medical intervention related to a drug”.

While ADEs are often a result of a ME, not all ADEs are associated with MEs. ADEs can be further divided into categories including preventable vs. non-preventable, ameliorable vs non-ameliorable and potential vs. actual.

MEs occur significantly more frequently than ADEs. Both have been associated with significant patient morbidity, mortality and cost. For example, it has been estimated that in the U.S., a hospital patient experiences one ME per day, and that 1/20 hospital inpatients experiences an ADE. MEs have also been estimated to contribute to 7000 deaths per year, and cost the healthcare system $21 billion annually.

A key step in minimizing MEs is performing checks for the medication, before its administration, against the patient, known patient allergies, other medications which the patient is currently taking or prescribed, and patient comorbidities and/or contra-indications.

Technology presents a significant opportunity to mitigate MEs and ADEs in an era of increasing patient acuity, complex workflows, and escalating clinician burnout. An electronic medication order presents one opportunity to perform a basic safety check for each prescribed medication. Typically, a Physician creates a medication order using a CPOE (or Computerized Physician Order Entry) system. CPOE systems have access to a patient's allergies, other prescribed/administered medications, and some of the patient comorbidities/factors which might present contra-indications to the medication the Physician intends to prescribe. CPOE systems conduct this check automatically and provide an opportunity for the Physician to review the results prior to e-signing an order. After reviewing these results, the Physician may decide to eliminate the order, prescribe a different medication or to weigh the benefits and risks and proceed with the originally intended medication.

CPOE is precluded in certain clinical workflows such as during medication administrations in most procedural settings (e.g. Operating Rooms, Cardiac Cath labs etc.). Here, CPOE systems are deemed to not be able to keep pace with complex clinical workflows. The immediate presence of physicians and/or advanced practice providers is also construed as mitigating the need to use CPOE. However, a recent study indicated that 1/20 medication administrations in an Operating Room at a large teaching hospital were associated with an ME, with 1/2 patients having surgery affected. Patient emergencies in any clinical setting (ICU, PACU, Emergency Room, Floor etc.) where time is deemed to be of essence also often preclude the use of CPOE. The prevalence of Verbal Orders and clinician burnout, can further reduce the efficacy of CPOE.

Bar Code Medication Administration (BCMA) systems present a second opportunity to mitigate ME/ADE. However, in a recent Leapfrog/Castlight Health survey, approximately 2/3 of all responding hospitals did not meet the Leapfrog National Standard for BCMA use due to deficiencies in integration with Electronic Health Records, and a lack of decision support tools.

While technologies such as CPOE (Computerized Physician Order Entry) and BCMA (Bar Code Medication Administration) systems have proven useful in multiple Hospital workflows; however, these technologies fall short in more complex clinical workflows (e.g. in the operating room or in the case of patient emergency).

To minimize MEs, the safety provided by both CPOE and BCMA must be augmented to meet the needs of current complex clinical workflows. At its core, for a solution to be effective, it must be fast enough to keep pace with a medication administration process when time is of the essence. It must also minimize nuisance alerts and cognitive overload, work across existing data siloes and seamlessly integrate into clinical workflows.

### SUMMARY OF THE INVENTION

The present invention is directed to a drug interaction checking tool that can be used without medication orders or CPOE which includes an interface sub-system to receive messages from healthcare information technology systems with information about the patient such as patient allergies, other medications the patient is currently taking, administered or prescribed to the patient, patient factors and co-morbidities, where the interface sub-system also receives messages from a decentralized pharmacy system with information about the withdrawn medications, the clinician who withdrew the medications, and the patient for whom the medications are intended; a data repository sub-system to store the received data such as patient allergies, medications prescriber, administered or currently in use, patient factors and co-morbidities, medications withdrawn for the patient, and the identity of the clinician who withdrew the medications; access to a medication interactions database; a guidance engine sub-system which performs the medication interaction checks for the patient and generates alerts if a risk of Medication Errors or adverse reaction is identified; and a notification sub-system which disseminates the alerts to one or more devices/systems such as the clinician's mobile device, the clinician's wearable device (e.g. smartwatch), a dashboard screen, a point of care sub-system, a paging system, a messaging system, and the like.

### DESCRIPTION OF THE DRAWINGS

The foregoing and other features and advantages of the present invention will become apparent to those skilled in the art to which the present invention relates upon reading the following description with reference to the accompanying drawings, in which:

FIG. 1 is a diagram of a global view of the present invention and other systems with which it interacts;

FIG. 2 is a diagram of the present invention sub-system showing interactions between invention sub-systems and between an invention sub-system and other systems external to the invention;

FIG. 3 is a sequence diagram showing the sequence of interactions between the Clinician end user and the invention/other systems, as well as the sequence of interactions between the invention and other systems external to the invention as described in Example 1;

### DETAILED DESCRIPTION OF THE INVENTION

The present invention fills the gap not fulfilled by systems such as CPOE and BCMA to meet the needs of complex clinical workflows. The present invention presents a significant opportunity to mitigate MEs (Medication Errors) and ADEs (Adverse Drug Events) in an era of increasing patient acuity, complex workflows, and escalating clinician burnout.

The present invention is directed to medication administration process (or method) and tool (or system) which is especially applicable to drugs administered without a medication order. The system and method of the present invention is effective for the identified patient regardless of location and any decentralized pharmacy station can be supported. User interactions (e.g. notification alerts) can be via any number of mobile devices including but not limited to tablets, smart phone, and/or wearable devices including but not limited to smart watches, and the like.

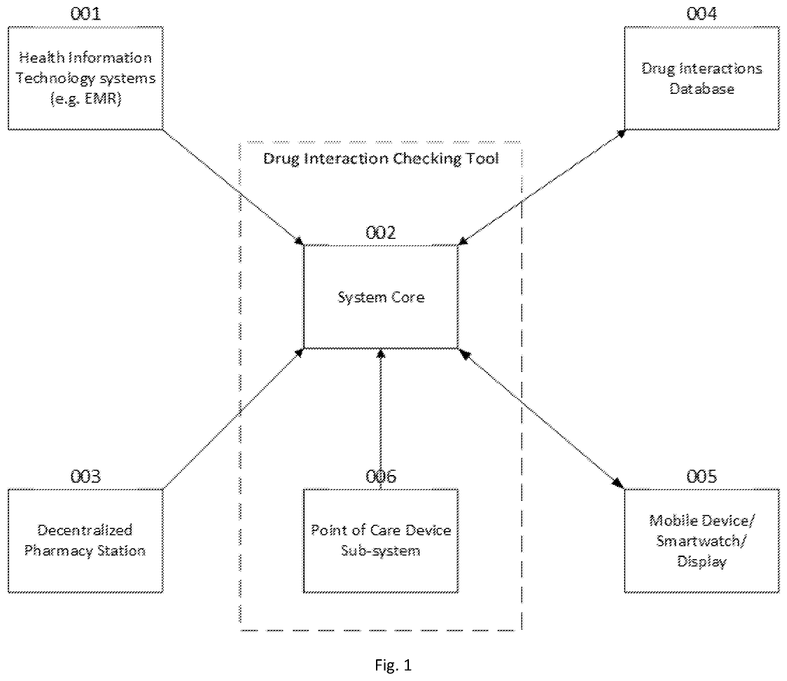

As can be seen in FIG. 1, the method of the present invention involves identifying the medications intended for administration as early as possible, rapidly checking against allergies, other medications, contra-indications and/or co-morbidities, and other patient factors, and notifying the clinicians as soon as possible before they get a chance to administer the medication, by a (Clinical Decision Support) notification which goes to the Clinician's personal mobile devices and wearables, and/or other appropriate POC (point of care) sub-systems where the correct patient may be, and sending guidance to a dashboard to support notification.

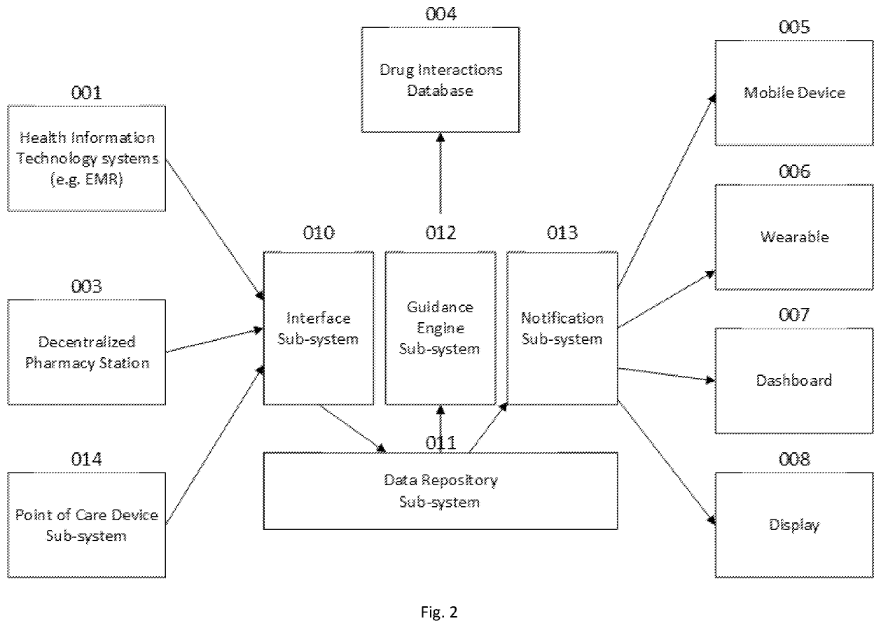

As seen in FIGS. 1 and 2, the present invention includes an interface sub-system 010, such as is described in co-pending U.S. patent application Ser. No. 16/238,187, filed Jan. 2, 2019, the disclosure of which is incorporated herein by reference, to receive messages from healthcare information technology systems 001 with information about the patient such as patient allergies, other medications the patient is currently taking, administered or prescribed to the patient, patient factors and co-morbidities. This interface sub-system 010 also receives messages from a decentralized pharmacy station 003 with information about the withdrawn medications, the clinician who withdrew the medications, and the patient for whom the medications are intended; a data repository sub-system 011 to store the received data such as patient allergies, other medications, patient factors and co-morbidities, medications withdrawn for the patient, and the identity of the clinician who withdrew the medications; access to a medication interactions database 004; a guidance engine sub-system 012 which performs the medication interaction checks for the patient and generates alerts if a risk of a Medication Error or adverse reaction is identified; and notification sub-system 012 which disseminates the alerts to one or more devices/systems 005, 006, 007, 008 such as the clinician's mobile device (e.g. smartphone, tablet), the clinician's wearable device (e.g. smartwatch), a dashboard screen, a paging system, a messaging system, a point of care sub-system 006 and the like.

In the system and method of the present invention the system of the present invention receives patient information such as allergies, other medications, patient factors and co-morbidities from Healthcare Information Technology systems 001 such as an Electronic Health Record (EHR) or Personal Health Record (PHR) system. Additionally, a Clinician can use the invention's point-of-care sub-system 006 to capture a barcode scan of the patient's wristband. Because the invention's point-of-care sub-system 006 is pre-configured with a fixed location (e.g. operating room 5), the invention can associate the patient with the location. The Clinician identifies themselves by logging into the decentralized pharmacy station 003. The Clinician identifies the patient for whom the medications are intended to be administered and the identified medication(s) to be withdrawn. The Clinician withdraws one or more medications from the decentralized pharmacy station 003. The system of the present invention receives transactions for each withdrawn medication. The system uses a drug interactions database 004 to automatically perform the following checks:

a) Drug-to-Allergy interaction by checking withdrawn medication (and its medication class) against patient allergies received from the healthcare information technology systems.

b) Drug-to-Drug interaction by checking withdrawn medication (and its medication class) against other medications (and medication classes) the patient is currently taking, administered or prescribed as received from the healthcare information technology systems

c) Drug-to-Patient interaction by checking withdrawn medication (and its medication class) against patient factors (e.g. pregnancy) and co-morbidities as received from the healthcare information technology systems.

The process notifies the Clinician who withdrew the medication immediately of any risk of adverse reaction to the medication taken from the decentralized pharmacy station and sends a message directly to the Clinician as soon as possible wherever they may physically be. To achieve that speed, the invention directs the notifications/alerts to the Clinician's mobile device and/or wearable device (e.g. smart watch).

The process can send a notification to the Point of Care 006. A display device located at the point-of-care can alert everyone at the point-of-care of a risk of Medication Error or adverse reaction to the medication taken from the decentralized pharmacy station 003. The process can send a notification to a supervising Physician. The process can send a notification to a Dashboard screen 007 indicating that a risk of Medication Error or adverse reaction to the medication taken from the decentralized pharmacy station 003 has been identified. When the barcode on the patient wristband is scanned associating the patient with the point-of-care location, the Dashboard 007 will display the location of the patient for whom the medications have been withdrawn.

The following are examples of Drug Interaction Checking Tool Without Medication Orders. They are described in the context of an anesthesiologist, but they could be applied to any clinician that has the authority to withdraw drugs without a medication order.

### Example 1

A patient undergoing surgery has a Cephalosporin allergy. The system of the present invention receives patient information from the healthcare information technology systems in the hospital. The patient's history of Cephalosporin allergy is received and stored by the invention.

During surgery, the anesthesiologist is in the operating room with the patient. The patient needs an antibiotic at this stage of the surgery.

The anesthesiologist turns to the de-centralized pharmacy station 003 inside the operating room and identify themselves in the system. The anesthesiologist identifies the patient for whom the medication is intended. The anesthesiologist choses to withdraw the antibiotic Cefazolin. The de-centralized pharmacy station 003 dispenses the Cefazolin medication. The de-centralized pharmacy system 003 sends a message to the system core 002 of the present invention including the following details:

Timestamp of the transaction

The identity of the clinician who withdrew the medication

The identity of the patient for whom the medication was drawn

The medication withdrawn for the patient (Cefazolin in this example)

In this type of clinical workflow, receiving such message from the de-centralized pharmacy system 003 is the earliest opportunity for the invention to know that the anesthesiologist intends to deliver a Cefazolin antibiotic. Receiving messages from the de-centralized pharmacy system 003 triggers the invention to execute a series of drug checks against previously received patient information.

When the drug-to-allergy check is executed, a risk is identified of an adverse reaction to administering Cefazolin due to a known interaction with the patient's Cephalosporin allergy per the drug interaction database 004. The system core 002 of the present invention, i.e., the drug interaction checking tool, generates an alert and immediately notifies the anesthesiologist of the risk of adverse reaction due to the drug-to-allergy interaction between the withdrawn Cefazolin antibiotic and the patient's recorded Cephalosporin allergy. A notification is sent to the anesthesiologist smartwatch or smartphone to maximize the probability that they see the alert notification prior to administering the medication. Once informed, the anesthesiologist relies on their education and knowledge to decide to either pick a different antibiotic or weigh the risks and benefits and proceed with administering the Cefazolin as originally planned.

### Example 2

As a further example, a patient undergoing surgery has a history of Malignant Hyperthermia disease. The system core 002 of the present invention receives patient information from the healthcare information technology systems 001 in the hospital. The patient history of Malignant Hyperthermia disease is received and stored by the system of the present invention.

When the patient is wheeled into the operating room, a certified registered nurse anesthetist barcode scans the patient wristband into the invention's point-of-care sub-system 006. The system core 002 of the invention associates the patient with the pre-identified location of the point-of-care sub-system 006 from which the patient wristband has been scanned.

During surgery, the anesthesiologist is not in the operating room with the patient, instead they are supervising multiple operating rooms where a certified registered nurse anesthetist is physically at each point-of-care.

The anesthesiologist, using the de-centralized pharmacy station 003 in the hallway outside the operating room, identifies themselves in the system, identifies the patient for whom the medication is intended, and chooses to withdraw the muscle relaxant Succinylcholine. The de-centralized pharmacy station 003 dispenses the Succinylcholine medication. The de-centralized pharmacy system sends a message to the system of the present invention including the following details:

Timestamp of the transaction

The identity of the clinician who withdrew the medication

The identity of the patient for whom the medication was drawn

The medication withdrawn for the patient (Succinylcholine in this example)

Receiving such message from the de-centralized pharmacy system 003 triggers the system core 002 of the present invention to execute a series of drug checks against previously received patient information. When the drug-to-disease check is executed, a risk is identified of an adverse reaction to administering Succinylcholine to a patient with a history of Malignant Hyperthermia disease per the drug interaction database 004.

The present invention generates an alert and immediately notifies the anesthesiologist of the risk of adverse reaction due to the drug-to-disease interaction between the withdrawn Succinylcholine muscle relaxant and the patient's recorded history of Malignant Hyperthermia. A notification is sent to the anesthesiologist smartwatch to maximize the probability that they see the alert notification prior to administering the medication. Additionally, the system core of the present invention sends a notification to a device/system at the point of care and the mobile device of the certified registered nurse anesthetist inside the operating room to notify them of the risk of adverse reaction and sends a notification alert to a dashboard 007 screen placed in the operating room control desk to notify other care givers of the risk of drug-to-disease adverse reaction in the patient's operating room.

Once informed, the anesthesiologist relies on their knowledge and experience to make a medical decision on how to proceed. The present invention provides a method to review a medication pulled from a decentralized pharmacy system against patient details, obtained from a health information system such as an EHR or PHR 001, utilizes a Drug Interaction Database 004 to query known risks associated with allergies, other medications, redosing, diseases, and patient factors, and provides rapid Clinical decision support recommendations based on this comparison. The present invention provides a mechanism for assessing data received from any number of sources within a global network and a mechanism to communicate required information to the appropriate individual(s) regardless of their current location and to the appropriate point of care.

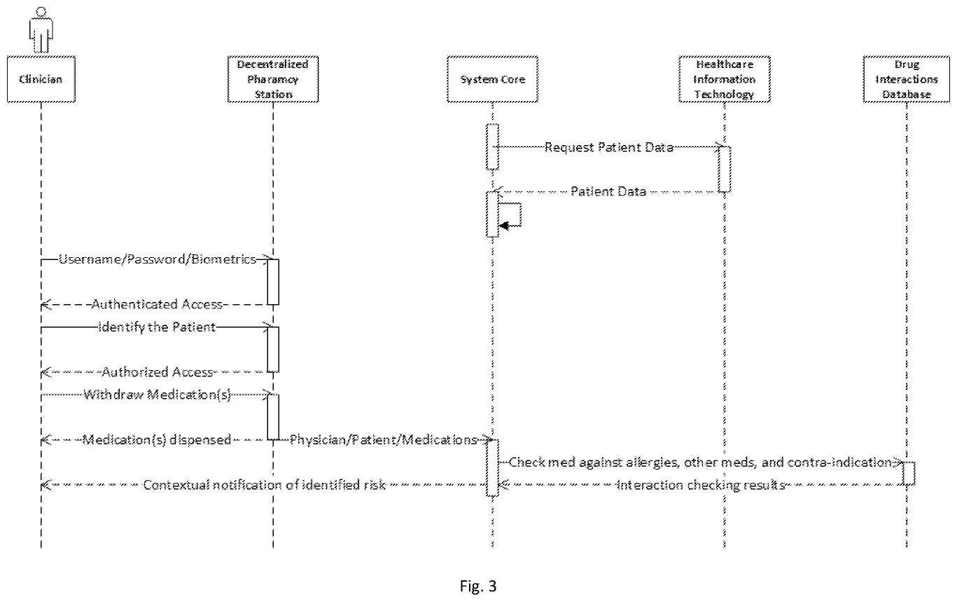

As seen in FIG. 3, the system of the present invention allows the reviewer to see and understand the relationship and order between the subsystems and interactions with the practitioners and subsystems external to the invention and how they are leveraged for analysis and notifications as described above. FIG. 3 demonstrates the value of the present invention in performing the analysis and notification in a timely fashion and its importance in managing the drug interaction issue prior to delivery in a time-sensitive situation.

The foregoing embodiments of the present invention have been presented for the purposes of illustration and description. These descriptions and embodiments are not intended to be exhaustive or to limit the invention to the precise form disclosed, and obviously many modifications and variations are possible in light of the above disclosure. The embodiments were chosen and described in order to best explain the principle of the invention and its practical applications to thereby enable others skilled in the art to best utilize the invention in its various embodiments and with various modifications as are suited to the particular use contemplated.

What we claim is:

1. A drug interaction checking tool without medication orders comprising:
an interface sub-system to receive messages from healthcare information technology systems with information about a patient such as patient allergies, medications the patient is currently taking, administered or prescribed to the patient, patient factors and co-morbidities; said interface sub-system further receiving messages from a decentralized pharmacy system with information about withdrawn medications, a clinician who withdrew the medications, and the patient for whom the medications are intended;
a data repository sub-system to store the received data such as patient allergies, medications administered, prescribed or currently in use, patient factors and co-morbidities, medications withdrawn for the patient, and the identity of the clinician who withdrew the medications;
access to a medication interactions database;
a guidance engine sub-system which performs medication interaction checks for the patient and generates alerts if a risk of Medication Error or adverse reaction is identified;
a notification sub-system which disseminates the alerts to one or more devices/systems such as a clinician's mobile device, the clinician's wearable device, a dashboard screen, a point of care sub-system a paging system, a messaging system, and the like.

2. The system defined in claim 1, wherein a point of care sub-system is used to accept a barcode scan of the patient wristband to be used by the invention for association between the patient and the location where the scan occurred.

3. The system defined in claim 1, wherein the guidance engine sub-system allows authorized clinicians, through a graphical user interface, to add more detailed rules beyond the simple interactions of the drug interaction database leveraging all available data collected, including device data, about the patient for whom the medications are withdrawn, the clinician who withdraws the medications, and the location associated with the patient.

4. A method for checking for drug interaction, comprising:
Providing a system core for receiving, storing and processing information about a patient;
obtaining and processing, by the system core, patient information from a health information technology system including an electronic health record or personal health record database;
receiving and processing, by the system core, a medication dispensing transaction;
reviewing, by the system core, the details of medication dispensed from a decentralized pharmacy station based on the received information as well as stored information about patient allergies, medications the patient has been administered, prescribed or is currently taking, patient factors and co-morbidities, and the identity of the clinician who withdrew the medication;
determining, by the system core, a guidance regarding medication interaction checks for the patient and generating an alert if a risk of medication error or adverse reaction is identified;
providing, by the system core via a communications module of the system core, information regarding risks identified with the administration of the dispensed medication; and
performing a notification of alerts to one or more devices/systems such as the clinician's mobile device, the clinician's wearable device, including a smartwatch, a dashboard screen, a paging system, point of care device sub-system and/or a messaging system.

5. A non-transitory machine-readable storage medium comprising machine-readable instructions for causing the system core to execute a method, the method comprising:
obtaining and processing, the system core, patient information from a health information technology system such as an electronic health record or personal health record database;
receiving and processing, by the system core, a medication dispensing transaction;
reviewing, by the system core, the details of medication dispensed from a decentralized pharmacy station based on the received information as well as stored information about patient allergies, medications the patient has been administered, prescribed or is currently taking, patient factors and co-morbidities, and the identity of the clinician who withdrew the medication;
determining, by the system core, a guidance regarding medication interaction checks for the patient and generating an alert if a risk of medication error or adverse reaction is identified;
providing, by the system core via a communications module of the system core, information regarding risks identified with the administration of dispensed medication; and
performing a notification of alerts to one or more devices/systems such as the clinician's mobile device, the clinician's wearable device, including a smartwatch, a dashboard screen, a paging system, a point of care sub-system and/or a messaging system.
